(19) United States
(12) Patent Application Publication
(10) Pub. No.: US 2014/0158788 A1
(43) Pub. Date: Jun. 12, 2014

(54) CONTROLLING A RELEASE OF AIRBORNE CHEMICALS

(71) Applicant: THE DIAL CORPORATION, Scottsdale, AZ (US)

(72) Inventors: Tyler R. Mikkelsen, Scottsdale, AZ (US); Kevin Hafer, Scottsdale, AZ (US); James Clark, Scottsdale, AZ (US)

(73) Assignee: The Dial Corporation, Scottsdale, AZ (US)

(21) Appl. No.: 13/707,656

(22) Filed: Dec. 7, 2012

(51) Int. Cl. A61L 9/12

(52) U.S. Cl. CPC A61L 9/12
USPC 239/6; 239/55

(57) ABSTRACT

Methods and apparatus are provided for controlling a release of airborne chemicals. The apparatus includes a wall thickness between an inside surface and an outside surface, and the inside surface is shaped to connect to a chemical source covering that releases an airborne chemical from a chemical source. The wall thickness also has multiple openings connecting the inside surface to the outside surface, and the openings occupy fifteen percent to forty five percent of the outside surface.

### FIELD OF THE INVENTION

The present invention generally relates to a device for releasing airborne chemicals, such as in an air freshener. More particularly, the present invention relates to a device that restricts an air flow to a chemical source with ventilation openings that cover fifteen percent to forty five percent of the device's exterior area.

### BACKGROUND OF THE INVENTION

Air fresheners remove or mask unpleasant odors in the air. Some types of air fresheners use a liquid or water based gel that contains a fragrance. When a protective seal to the liquid or gel is opened, the liquid or gel begins to evaporate over time, and the fragrance is released into the air. When all of the liquid or gel has evaporated, effective diffusion of the fragrance into the surrounding air will generally cease.

One condition in the ambient environment that affects the rate of evaporation is air flow to and around the liquid or gel containing the fragrance. Generally, a higher air flow will increase the evaporation rate, while a lower air flow rate produces a slower rate of evaporation. Thus, the evaporation rate changes as the air flow around the air freshener changes. Sudden gusts of air flow can produce corresponding spikes in the evaporation rate, such as when doors or windows to a room are opened or closed, or someone or something passes quickly by the air freshener. Sustained increases in air flow can similarly produce sustained increases in the evaporation rate. This may result from using a fan or other mechanism to circulate air, or other events that change the air flow in a room containing an air freshener.

Accordingly, it is desirable to control the air flow around air fresheners and other devices where a release of airborne chemicals, such as fragrances, is affected by air flow. In addition, it is desirable to increase the lifespan of such air fresheners and other devices by preventing their chemical sources from drying out prematurely. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An apparatus is provided for controlling a release of airborne chemicals. The apparatus includes a ventilation housing for placement over a chemical source covering. The ventilation housing has a wall thickness between an inside surface and an outside surface. The inside surface is shaped to connect to a chemical source covering that releases an airborne chemical from a chemical source. The wall thickness has multiple openings connecting the inside surface to the outside surface, and the openings occupy fifteen percent to forty five percent of the outside surface.

A method is provided for controlling a release of airborne chemicals. The method includes pulling on an external ventilation housing to open a chemical source covering connected to an inside surface of the external ventilation housing where the external ventilation housing has an outside surface with fifteen percent to forty five percent of its area containing ventilation openings to vent an airborne chemical released from the chemical source covering.

### BRIEF DESCRIPTION OF THE DRAWINGS

The present invention will hereinafter be described in conjunction with the following drawing figures, wherein like numerals denote like elements, and

FIG. 1 is an exploded diagram of an example of a chemical source covering and a ventilation housing according to the principles described herein.

FIG. 2 is a diagram of an example of a chemical source covering inserted into a ventilation housing according to the principles described herein.

FIG. 3 is a chart of an example of an evaporation rate of a chemical source according to the principles described herein.

FIG. 4 is a chart of an example of a lifespan of a chemical source according to the principles described herein.

FIG. 5 is a chart of an example depicting a chemical source's rate of evaporation in weight percentages according to the principles described herein.

FIG. 6 is a chart of an example depicting a chemical source's rate of evaporation based on placement of ventilation openings in a ventilation housing according to the principles described herein.

FIG. 7 is a chart of an example of a method for controlling a release of airborne chemicals according to the principles described herein.

FIG. 8 is a diagram of an example of a ventilation housing according to the principles described herein.

FIG. 9 is a diagram of an example of a ventilation housing according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As noted above, some types of air fresheners use a liquid or water based gel that contains a fragrance. The fragrance-bearing compound will also be referred to as a chemical source. When a protective seal to the chemical source is opened, the liquid or gel begins to evaporate over time, and the fragrance is released into the air.

Inconsistent evaporation rates due to varying amounts of air flow coming into contact with the chemical source lead to shortened air freshener life. Further, when the seal around an air freshener is initially broken, the air freshener's chemical source initially evaporates at a quicker rate than will occur several weeks later. Slowing the evaporation rate early in the air freshener's life saves more of the chemical source for later which provides a more consistent fragrance profile or output over the air freshener's life and can even extend the life of the air freshener. These same principles, and the subject matter disclosed herein, may also apply to other products that emit different airborne chemicals into the air through passive evaporation, such as pesticides, insecticides, insect repellants, animal repellants, other chemicals, or combinations thereof.

As described herein, a ventilation housing that restricts the air flow is placed around an original housing for a chemical source such that bursts of increased air flow are restricted from reaching the chemical source. This reduces the uneven air flow's effect on the chemical source's evaporation rate. However, the ventilation housing also permits enough air flow to reach the chemical source such that a desired amount of the airborne chemicals are still released into the ambient atmosphere. Consequently, the ventilation housing increases the linearity of the chemical source's evaporation rate which can lead to increased user satisfaction and, in some cases, increased product life.

The ventilation housing includes a wall thickness between an inside surface and an outside surface. The inside surface is shaped to cover or connect to an original housing or other chemical source covering from which release of an airborne chemical from a chemical source occurs. The wall thickness has multiple openings connecting the inside surface to the outside surface. The openings occupy fifteen percent to forty five percent of the outside surface.

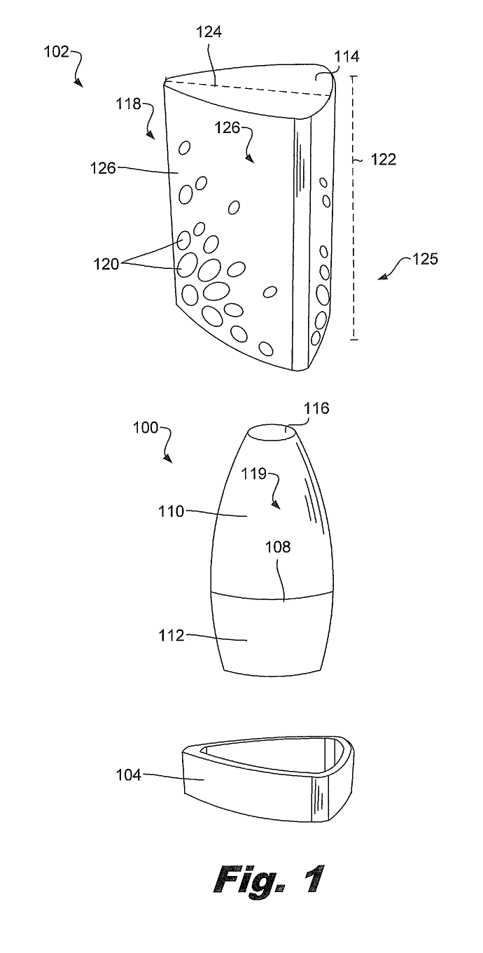

FIG. 1 is an exploded diagram of an example of a chemical source covering (100) and a ventilation housing (102). In this example, the chemical source covering (100) contains a chemical source, such as fragrances, repellants, pesticides, or other chemicals for consumer or industrial use. The chemical source covering (100) is insertable into a base (104) and an external, ventilation housing (102) is shaped to be placed over the top of the chemical source covering (100).

In the example of FIG. 1, the chemical source covering (100) is closed along a break joint (108) and thereby prevents the chemical source from evaporating and releasing airborne chemicals into the ambient environment. When the break joint (108) is broken, an upper portion (110) of the chemical source covering (100) lifts away from a lower portion (112). A gap between the upper and lower portions (110, 112) forms an opening in the covering (100) where the airborne chemicals are released due to evaporation of the chemical source.

In the illustrated example, an underside of a top (114) of the ventilation housing (102) connects and/or rests on the top (116) of the chemical source covering (100) such that the ventilation housing's sides (118) enclose the chemical source covering (100). The sides (118) extend past the covering's break joint (108), where the chemical source is released. As a consequence, when the break joint (108) is open, the airborne chemicals are first released into an envelope between the covering (100) and the ventilation housing (102). This envelope is bounded by the top (114) and sides (118) of the ventilation housing (102).

In other examples, an inside surface of the ventilation housing (102) connects with the outside surface (119) of the chemical source covering (100) such that the housing (102) and the covering (100) are locked together. In such examples, the ventilation housing (102) may be locked to the chemical source covering (100) by twisting the ventilation housing (102) into a locking position once the chemical source covering (100) is placed within the ventilation housing (102). When locked together, the chemical source covering (100) can be opened by pulling up on the ventilation housing (102). In other examples, such as illustrated in FIG. 1, the ventilation housing (102) is removed to open the chemical source covering (100) and then replaced over the chemical source covering (100) to control air flow within the envelope between the two.

Ventilation openings (120) formed in the ventilation housing (102) permit the airborne chemicals to vent into the ambient environment while limiting or restricting changes in air flow from reaching the chemical source. Thus, when in place, the ventilation housing (102) provides a first barrier that limits changes in air flow from reaching the chemical source. The chemical source covering provides a second barrier that restricts the air flow changes from reaching the chemical source. As a consequence, the combination of the ventilation housing (102) and the chemical source covering (100) provide a dual barrier that minimizes changes in the air flow from affecting the chemical source's evaporation rate. Consequently, the double barrier also reduces the amount of airborne chemicals that will be released at a given moment. As a result, when the chemical source covering (100) is initially opened the double barriers restrict the amount of airborne chemicals that are initially released into the ambient environment and thereby preserve more of the chemical source for evaporation later in the product's life.

The ventilation openings (120) are distributed along the length (122) and width (124) of the ventilation housing (102). In FIG. 1, a majority of the ventilation openings (120) are concentrated within a bottom half (125) of the ventilation housing (102). The ventilation openings (120) may occupy fifteen percent to forty five percent of the area formed by the ventilation housing's outside surface (126). In some examples, the ventilation openings (120) occupy twenty percent to forty percent of the area formed by the ventilation housing's outside surface (126). The ventilation openings (120) may occupy any appropriate percentage of the area defined by the perimeter of the sides of the ventilation housing between fifteen percent and forty five percent.

The ventilation openings (120) may be of any appropriate shapes and sizes that collectively occupy fifteen percent to forty five percent of the ventilation housing's outside area (126). The outside area (126) is the entire area bounded by the parameters of the ventilation housing's outside surface (126). Further, the ventilation housing (102) may be any appropriate shape or length that houses the chemical source covering (100) and restricts air flow from affecting the evaporation rate of the chemical source when the chemical source covering (100) is open. The chemical source covering (100) may also be any appropriate size and shape.

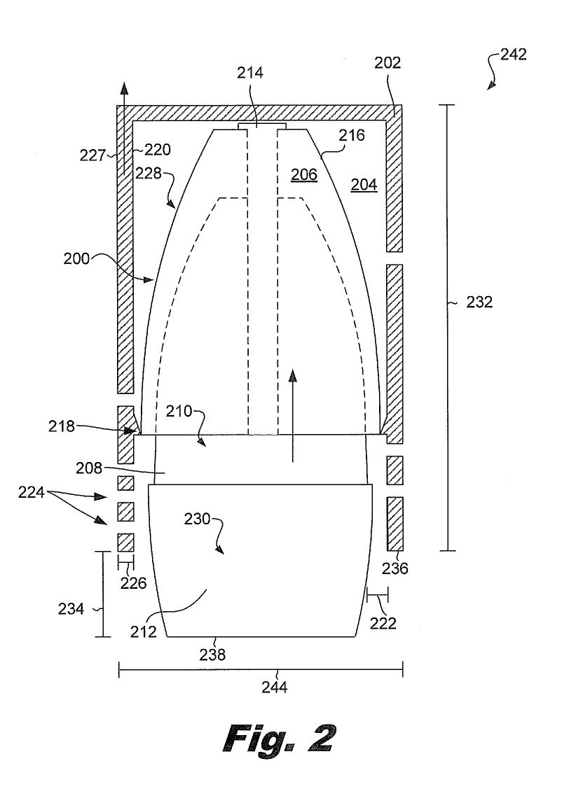

FIG. 2 is a diagram of an example of a chemical source covering (200) inserted into a ventilation housing (202). In this example, an inner cavity (204) of the ventilation housing (202) receives and is connected and locked to the upper portion (206) of the chemical source covering (200). Consequently, as the ventilation housing (202) is pulled up, the upper portion (206) of the chemical source covering (200) is lifted away from the lower portion (212) such that a release opening (210) or gap is formed between the upper portion (206) and the lower portion (212) of the chemical source covering (200). As the upper portion (206) is lifted, the release opening (210) increases and exposes more of the chemical source (208) to an inner cavity (204) of the ventilation housing (202). The ventilation openings (224) formed in the ventilation housing (202) permit the airborne chemicals to pass from the inner cavity (204) of the ventilation housing (202) to an ambient environment outside of the ventilation housing (202).

A stopper (214) built into the chemical source covering (200) limits the upward range of the upper portion (206) of the covering (200) and thus controls the maximum size of the release opening (210). In some examples, the release opening (210) is adjustable to control the rate of evaporation of the chemical source. For example, the rate of passive evaporation is increased as the release opening (210) is enlarged. Likewise, the rate of passive evaporation decreases as the release opening (210) is decreased. Thus, within other variable at play, a user may adjust the rate at which airborne chemicals are released into the ambient environment by adjusting the size of the release opening (210) formed in the chemical source covering (200).

In the illustrated example, the inside surface (220) of the inner cavity (204) has at least one locking feature (218) that connects with the outside surface (216) of the upper portion (206) of the chemical source covering (200). In some examples, the locking feature (218) is part of a snapping mechanism that snaps into a recess formed in the upper portion (206) of the chemical source covering (200). In other examples, the locking feature (218) is part of a twisting mechanism where the locking feature threads or interlocks with the upper portion (206) of the chemical source covering (200) when the ventilation housing (202) is turned relative to the chemical source covering (200).

In yet other examples, the ventilation housing (202) locks to the chemical source covering (200) through a compression fit. In such examples, the locking features (218) squeeze the outside surface (216) of the chemical source covering (200) enough that the ventilation housing (200) and the upper portion (206) of the chemical source covering (200) are held together through friction. The compression fit may also be accomplished with an appropriately sized ventilation housing (202) without a locking feature. In such an example, the inner cavity (204) has a cross section that is slightly smaller than a widest portion of the outside surface (216) of the upper portion (206) of the chemical source covering (200). As the chemical source covering (200) is inserted into the ventilation housing (202), the walls of the ventilation housing flex outward to accommodate the widest portion of the upper portion (206) creating a compression fit that locks the ventilation housing (202) and the chemical source covering (200) together through friction.

Thus, various locking mechanisms, both with and without a locking feature formed on the housing (202) or covering (200) have been described. Any appropriate locking mechanism that causes the upper portion (206) of the chemical source covering (200) to move with the ventilation housing (202) may be used.

In the illustrated example, the release opening (210) is positioned near some of the ventilation openings (222) of the ventilation housing (202). In some examples, the release opening (210) is positioned within a distance (222) of less than twenty five millimeters of the ventilation openings (224). In other examples, the distance (224) is less than twenty millimeters from the ventilation openings (224). In yet other examples, the release opening (210) is positioned any appropriate distance from the ventilation openings (224). Initial testing has shown that the ventilation housing (202) was less effective for releasing the airborne chemicals outside into the ambient environment when the release opening (210) was positioned twenty millimeters or more from the ventilation openings (224).

The ventilation housing (202) has a wall thickness (226) between the inside surface (220) and an outside surface (227). The ventilation openings (224) are formed in the wall thickness (226) and communicate between the inside surface (220) to the outside surface (227) of the ventilation housing (202). Any of the ventilation openings (224) may be formed in any appropriate location in the ventilation housing (202) including in the upper half (228) and the bottom half (230) of the ventilation housing (202). In some examples, the bottom half (230) of the ventilation housing (202) includes a majority of the ventilation openings (224). Consequently, a majority of the ventilation exposure provided by the ventilation openings (224) occurs over the bottom half (230) of the ventilation housing (202). The ventilation exposure includes the summation of all the area occupied by the ventilation openings (224).

The ventilation housing (202) may have any appropriate dimensions. In some examples, the length (232) of the ventilation housing (202) is between five and six inches. The distance (234) between a bottom (236) of the ventilation housing (202) and a bottom (238) of the chemical source covering (200) may be between one and two inches. In such examples, the overall height of the system (242) is between six and eight inches. The width (244) of the ventilation housing (202) may be between three and four inches. The chemical source (208) may between seven and ten fluid ounces.

When the chemical source (208) is depleted, the chemical source covering (200) can be removed from the ventilation housing (202) by pulling on the ventilation housing (202) with sufficient force to overcome the locking mechanism that secures the ventilation housing (202) to the chemical source covering (200). In other examples, the ventilation housing (202) is unlocked from the chemical source covering (200) through a reverse twisting action thereby making the chemical source covering (200) removable. However, any appropriate removing mechanism may be used to remove the ventilation housing (202) from the chemical source covering (200). In some examples, the used chemical source covering (200) is discarded or recycled. Once removed, the ventilation housing (202) may be reused to house another chemical source covering (200).

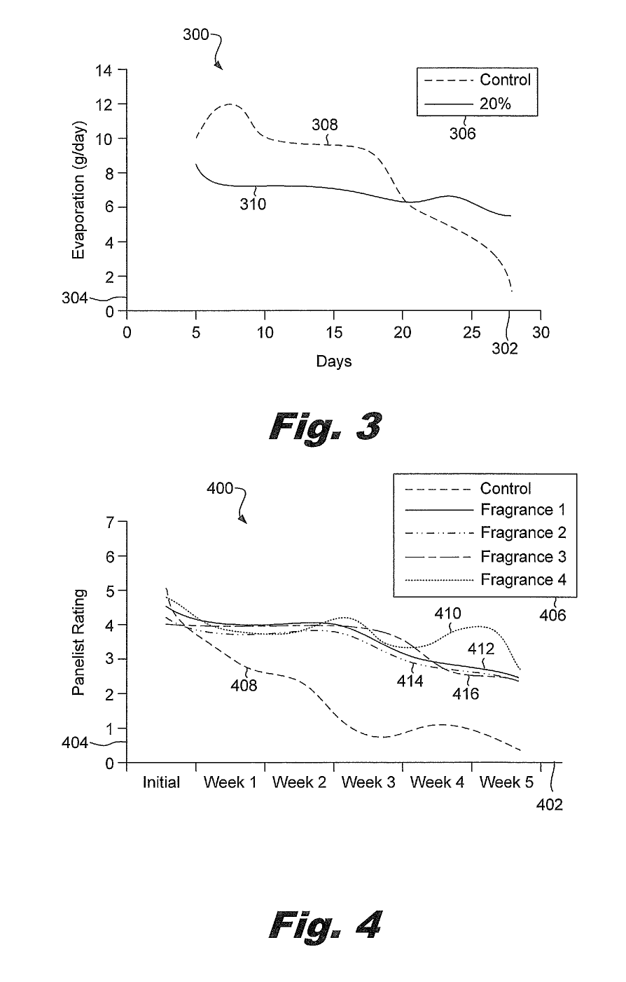

FIG. 3 is a chart (300) of an example of an evaporation rate of a chemical source. In this example, the x-axis (302) schematically represents a number of days, and the y-axis (304) schematically represents an evaporation rate. The evaporation rate is measured by weighing the chemical source on a periodic basis. As the chemical source evaporates, the chemical source weighs less. Thus, a chemical source that loses more grams per day has a higher rate of evaporation. A legend (306) indicates that the dashed line (308) schematically represents a control air freshener that does not contain the ventilation housing. The legend (306) also indicates that the solid line (310) schematically represents an air freshener with a ventilation housing that has ventilation openings that occupy twenty percent of the ventilation housing's outside surface.

In this example, the control air freshener and the air freshener with the ventilation housing were measured after five days. The control air freshener, represented with the dashed line (308), exhibited a higher evaporation rate than the air freshener with the ventilation housing. The control air freshener continued to exhibit a higher evaporation rate until about the seventeenth day, at which point, the evaporation rate significantly declines until the measurements were discontinued. The evaporation rate for the control air freshener ranged from about twelve grams per day to one gram per day. Further, the control air freshener's rate of evaporation significantly declined over its life span.

On the other hand, the air freshener with the ventilation housing exhibited an evaporation rate range between about six grams and eight grams per day. Also, the evaporation rate for the air freshener with the ventilation housing exhibited a much more consistent evaporation rate throughout the test. While the control air freshener appears to have exhausted most of its chemical source at about the twenty seventh day, the air freshener with the ventilation housing appears to have a substantial amount of chemical source remaining which may allow it to have a useful life that extends beyond the twenty seventh day.

FIG. 4 is a chart (400) of an example of a lifespan of a chemical source. In this example, the x-axis (402) schematically represents a number of weeks, and the y-axis (404) schematically represents a panelist rating. The panelist rating is determined by a group of panelists who were asked to rate their satisfaction of each air freshener used each week during the duration of the test. A legend (406) indicates that the dashed line (408) schematically represents a control air freshener that does not contain the ventilation housing. The legend (406) also indicates that the other lines (410, 412, 414, 416) schematically represent air fresheners with ventilation housings that use chemical sources with different types of fragrances.

The chart (400) indicates that each of the air fresheners with ventilation housings achieved a much higher satisfaction rating from the panelists over the control air freshener regardless of the fragrance type. In this example, the control air freshener's satisfaction rating was similar to the other air fresheners when the panelist's were initially questioned at the beginning of the test. However, after the first week, the control air freshener's satisfaction rating significantly dropped. After the second week, the control air freshener's satisfaction rating was below a two of out seven, which indicates that the control air freshener performed poorly. On the other hand, the other air fresheners with ventilation housings initially achieved a satisfaction rating of about a four to five and at the end of the test, in the fifth week, were still achieving a satisfaction rating of about three. Thus, regardless of the different types of air fresheners' fragrances, each air freshener with a ventilation housing significantly outperformed the control air freshener for a significantly longer time period.

Any appropriate fragrance may be used in an air freshener made in accordance with the principles described herein. For example, compatible fragrance types may include fragrances from the floral family, the oriental family, the citrus family, the chypre family, the green family, the fougere family, other types of fragrances, or combinations thereof.

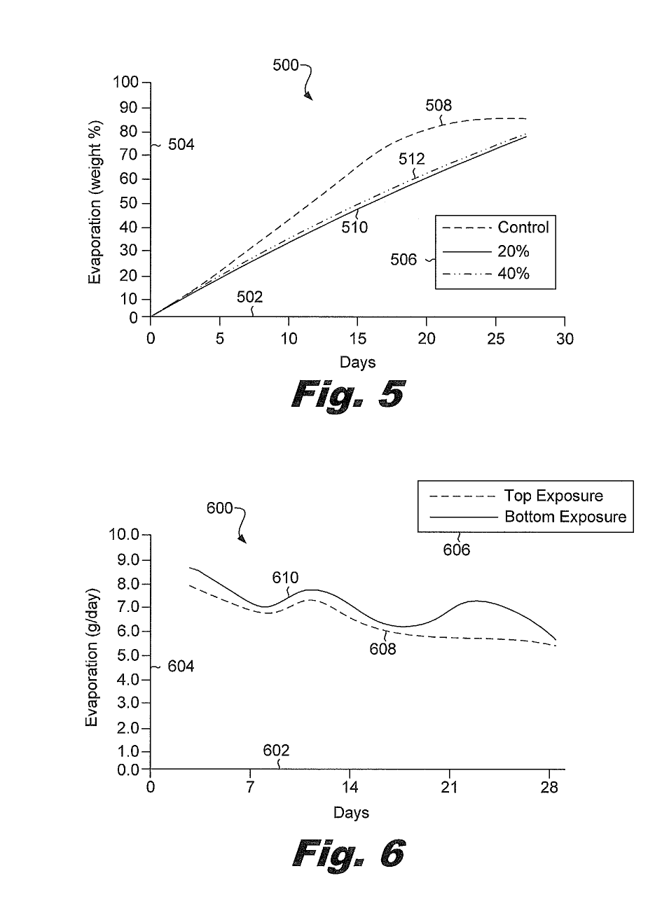

FIG. 5 is a chart (500) of an example depicting a chemical source's rate of evaporation in weight percentages. In this example, the x-axis (502) schematically represents a number of days, and the y-axis (504) schematically represents a percentage of the chemical source that has evaporated away. A legend (506) indicates that the dashed line (508) schematically represents a control air freshener that does not contain the ventilation housing. The legend (506) also indicates that line (510) schematically represents an air freshener with a ventilation housing that has ventilation openings that occupy twenty percent of the ventilation housing's outside surface. Further, the legend (506) also indicates that line (512) schematically represents an air freshener with a ventilation housing that has ventilation openings that occupy forty percent of the ventilation housing's outside surface.

The chart (500) indicates that the control air freshener evaporated more rapidly than both the air fresheners with ventilation housings, schematically represented with lines (510, 512). At twenty days, nearly ninety percent of the control air freshener's chemical source was evaporated away, while both of the other air fresheners having ventilation housings had just sixty percent of their chemical sources evaporated away.

FIG. 6 is a chart (600) of an example depicting a chemical source's rate of evaporation based on placement of the ventilation openings in a ventilation housing. In this example, the x-axis (602) schematically represents a number of days, and the y-axis (604) schematically represents an evaporation rate. A legend (606) indicates that the dashed line (608) schematically represents an air freshener with a ventilation housing with a majority of the ventilation exposure being located in the upper half of the ventilation housing surface. The legend (606) also indicates that the solid line (610) schematically represents an air freshener with a ventilation housing with a majority of the ventilation exposure being located in the bottom half of the ventilation housing surface.

The testing indicates that air fresheners with the ventilation exposure being concentrated towards the bottom half of the ventilation housing has a lower evaporation rate than the air freshener with the ventilation exposure being concentrated in the upper half of the ventilation housing. Further, the air freshener with the ventilation exposure concentrated in the bottom half of the ventilation housing exhibited a more linear evaporation rate.

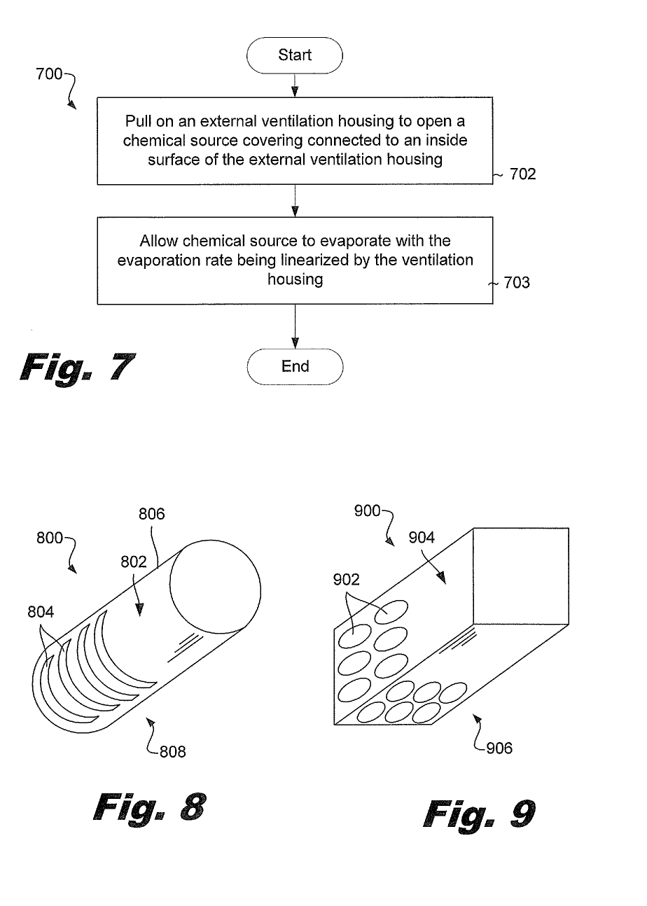

FIG. 7 is a flowchart of an example of a method (700) for controlling a release of airborne chemicals. In this example, the method (700) includes pulling (702) on an external ventilation housing to open a chemical source covering connected to an inside surface of the external ventilation housing. In the illustrated example, the external ventilation housing has an outside surface with fifteen to forty five percent of its surface area containing ventilation openings to vent an airborne chemical released from the chemical source covering. After opening, the method concludes with allowing (703) the chemical source to evaporate with the evaporation rate being linearized by the ventilation housing.

In some examples, this method might also include inserting the chemical source covering into the inside surface of the external ventilation housing and replacing the chemical source with a new chemical source when the original is depleted. As disclosed above, the external ventilation housing may have the characteristic of restricting air flow around the chemical source such that inconsistencies in an evaporation rate of the chemical source are reduced. In the illustrated example, the chemical source covering is positioned less than twenty five millimeters away from the ventilation opening. The ventilation openings are distributed throughout the outside surface. A bottom half of the external ventilation housing includes a majority of the ventilation.

FIG. 8 is a diagram of an example of a ventilation housing (800). In this example, the ventilation housing (800) has a cylindrical shape (802) and multiple ventilation openings (804). The ventilation openings communicate between an inside surface of the ventilation housing (800) and the outside surface (806) of the ventilation housing (800). A chemical source covering can be inserted into an inner cavity formed by the ventilation housing's inside surface. In this example, the ventilation openings (804) are formed as slots. Each of the slots are aligned in a row and all of the slots are positioned in a bottom half (808) of the ventilation housing (800).

FIG. 9 is a diagram of another example of a ventilation housing (900). In this example, the ventilation housing (900) has ventilation openings (902) that have a circular shape. Further, the ventilation housing (900) has a rectangular shape (904). In this example, all of the ventilation openings (902) are positioned in the bottom half (906) of the ventilation housing (900).

While the examples above have been described with reference to specific shapes of ventilation housings and ventilation openings, any appropriate shape for both the ventilation housings and ventilation openings may be used in accordance with the principles described herein. While the ventilation openings are shaped to control the evaporation of the chemical source, the ventilation openings can also be shaped to provide an aesthetic look. In some examples, the ventilation openings are formed throughout the upper and bottom portions of the ventilation housing, and the ventilation openings get progressively larger towards the bottom of the ventilation housing. Also, while the examples above have been described with reference to specific distributions of the ventilation openings throughout the ventilation housing's outside surface or specific ventilation opening patterns, any appropriate distribution and/or pattern may be used in accordance with the principles described herein.

Any appropriate chemical source covering may be used to hold the chemical source. While the examples above have been described with specific reference to a chemical source covering with a release opening positioned circumferentially along the length of the covering, the release opening may be positioned on a single side, contiguous sides, multiple sides, tops, bottoms, other locations or geometries formed in a chemical source covering, or combinations thereof. Further, the shape and size of the release opening may be any appropriate size or shape. While the examples above have been described with specific reference to an adjustable release opening, the principles described herein include release openings that are fixed in size and/or shape.

Further, the chemical source covering may include multiple openings to collectively form a release opening. The chemical source covering may be any appropriate shape including cone shapes, rectangular shapes, triangular shapes, cylindrical shapes, symmetric shapes, asymmetric shapes, spherical shapes, aesthetic shapes, other shapes, or combinations thereof. Also, the ventilation housing may be any appropriate shape including cone shapes, rectangular shapes, triangular shapes, cylindrical shapes, symmetric shapes, asymmetric shapes, spherical shapes, aesthetic shapes, other shapes, or combinations thereof.

While the examples above have been described with reference to specific types of systems that release airborne chemicals into an ambient environment, any appropriate type of system that releases airborne chemicals may be used. For example, air fresheners that rely solely on passive evaporation may be used. However, active air fresheners may also incorporate the principles described herein. Active air freshener may include heating mechanisms that increases the chemical source's rate of evaporation when heat is applied. However, heating mechanisms or other active release mechanisms can be used in conjunction with the ventilation housing to more precisely control the rate of evaporation.

The examples above have been described with specific reference to liquid and water based chemical sources. However, any appropriate type of chemical source may be used. For example, oil based chemical sources, other types of chemical sources, or combinations thereof may also be used according to the principles described herein.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A ventilation housing for controlling a release of airborne chemicals, comprising:
a wall thickness between an inside surface and an outside surface;
said inside surface shaped to connect to a chemical source covering that releases an airborne chemical from a chemical source;
said wall thickness comprising a plurality of openings connecting said inside surface to said outside surface; and
said plurality of openings occupy fifteen percent to forty five percent of said outside surface.

2. The housing of claim 1, wherein said plurality of openings has a characteristic of restricting air flow around said chemical source such that inconsistencies in an evaporation rate of said chemical source are minimized.

3. The housing of claim 1, wherein said plurality of openings is distributed throughout said outside surface.

4. The housing of claim 3, wherein a majority of ventilation exposure formed with said plurality of said openings is in a bottom half of said ventilation housing.

5. A system for controlling a release of airborne chemicals, comprising:
a chemical source positioned within a source covering;
said source covering shaped to reside within an external ventilation housing;
said source covering comprising a release opening positioned to release an airborne chemical of said chemical source into said external ventilation housing; and
said external ventilation housing comprising at least one ventilation opening that connects an inside surface of said external ventilation housing to an outside surface of said external ventilation housing;
wherein said at least one ventilation opening comprises fifteen percent to forty five percent of an area of said outside surface.

6. The system of claim 5, wherein said external ventilation housing has a characteristic of restricting air flow around said chemical source such that inconsistencies in an evaporation rate of said chemical source are minimized.

7. The system of claim 5, wherein said airborne chemical is a fragrance.

8. The system of claim 5, wherein said airborne chemical is an pesticide, an insecticide, an insect repellent, an animal repellent, or combinations thereof.

9. The system of claim 5, wherein said chemical source covering is positioned less than twenty five millimeters away from said at least one ventilation opening.

10. The system of claim 5, wherein said release opening is adjustable to adjust a rate of passive evaporation of said chemical source.

11. The system of claim 10, wherein said release opening is vertically adjustable such that a section of said source covering lifts away from said chemical source as said release opening exposes more of said chemical source to an inner cavity of said external ventilation housing.

12. The system of claim 5, wherein at least one ventilation opening comprises a plurality of openings distributed throughout said outside surface.

13. The system of claim 12, wherein a majority of ventilation exposure formed with said plurality of said openings is in a bottom half of said external ventilation housing.

14. The system of claim 5, wherein said chemical source is a water based gel.

15. A method for controlling a release of airborne chemicals, comprising:
pulling on an external ventilation housing to open a chemical source covering connected to an inside surface of said external ventilation housing where said external ventilation housing comprises an outside surface with fifteen percent to forty five percent of its area containing ventilation openings to vent an airborne chemical released from said chemical source covering.

16. The method of claim 15, further comprising inserting said chemical source covering into said inside surface of said external ventilation housing.

17. The method of claim 15, wherein said external ventilation housing has a characteristic of restricting air flow around said chemical source such that inconsistencies in an evaporation rate of said chemical source are minimized.

18. The method of claim 15, wherein said chemical source covering is positioned less than twenty five millimeters away from said ventilation openings.

19. The method of claim 15, wherein said ventilation openings include a plurality of openings distributed throughout said outside surface.

20. The method of claim 19, wherein a majority of ventilation exposure formed with said ventilation openings is in a bottom half of said external ventilation housing.
